(19) United States
(12) Patent Application Publication
(10) Pub. No.: US 2023/0233520 A1
(43) Pub. Date: Jul. 27, 2023

(54) COMBINATION THERAPY OF ARTEMISININ-RELATED COMPOUNDS AND HISTONE DEACETYLASE INHIBITORS FOR TREATMENT OF HPV-RELATED BENIGN, PREMALIGNANT, AND MALIGNANT DISEASES

(71) Applicant: Georgetown University, Washington, DC (US)

(72) Inventors: Richard Schlegel, Rockville, MD (US); Hang Yuan, Washington, DC (US)

(73) Assignee: Georgetown University, Washington, DC (US)

(21) Appl. No.: 17/919,726

(22) PCT Filed: Apr. 23, 2021
(86) PCT No.: PCT/US2021/028864

§ 371 Date: Oct. 18, 2022

Related U.S. Application Data
(60) Provisional application No. 63/014,554, filed on Apr. 23, 2020.

(51) Int. Cl. A61K 31/357; A61K 31/4045; A61K 31/167; A61K 9/00; A61P 35/00; A61P 15/00

(52) U.S. Cl. CPC A61K 31/357; A61K 31/4045; A61K 31/167; A61K 9/0014; A61P 35/00; A61P 15/00

(57) ABSTRACT

Methods of treating human papillomavirus (HPV)-induced conditions, HPV-induced lesions, or HPV-infected cells. The method involves administering one or more artemisinin-related compounds and one or more histone deacetylase (HDAC) inhibitors. In addition, treatment regimens involving the use of artemisinin-related compounds and HDAC inhibitors, and kits comprising pharmaceutical compositions of artemisinin-related compounds and HDAC inhibitors.

### CROSS REFERENCE TO RELATED APPLICATION

This application claims priority to U.S. Provisional Application No. 63/014,554, filed Apr. 23, 2020, which is incorporated by reference herein in its entirety.

### FIELD OF THE DISCLOSURE

The present invention relates to the field of treatments of diseases or conditions associated with human papillomavirus. The present invention also relates to a combination therapy of artemisinin-related compounds and histone deacetylase inhibitors for use in such treatments.

### BACKGROUND

Cervical cancer is the second most common cancer in women in the developing world and the fourth most common cancer worldwide. With a global mortality rate of 52%, this illness has become a serious public health concern [1]. Past research has focused on elucidating potential cause(s) of cervical cancer, with the end goal being the identification of potential targets to treat or prevent the onset of the cancer. These studies have shown that 70% of cervical cancers are caused by two types of human papillomavirus (HPV), HPV 16 and HPV 18 [1]. Not all forms of HPV are linked to cancer—HPV 6 and HPV 11 are associated with the sexually transmitted disease that results in the formation of benign genital warts, and other types may cause no symptoms and are easily cleared by the immune system [1]. However, HPV 16, HPV 18, and the other high-risk HPVs are able to induce the formation of cancer because of their enhanced ability to integrate their DNA genomes into host cells and direct the expression of two especially potent viral oncoproteins, E6 and E7, which can directly bind to and block the activity of two integral host tumor suppressor proteins, p53 and pRb, respectively [2]. Thus, if the host immune system is unable to eliminate these virions and HPV-infected cells from the body, infection with these high-risk types will eventually promote the transition of healthy cells to cancer cells. When this process occurs in the cervical area, these high risk infections lead to premalignant lesions and then to malignant cervical cancer [3]. However, HPV is also responsible for the majority of other anogenital cancers including those at vaginal, vulvar, and anal sites, as well as cancers of oral, oropharyngeal and laryngeal origin.

One of the best ways to reduce the high mortality rate imposed by cervical cancer and other cancers associated with HPV is to intervene as early as possible, before the development of neoplastic (fully malignant) cells, because these cell types are the most difficult to target and kill. Currently, two FDA-approved vaccines exist to prevent HPV infection: GARDASIL® and CEVARIX™. For example, the administration of GARDASIL® 9 can protect against HPV 6, 11, 16, 18, 31, 33, 45, 52, and 58 infection, and thus is very effective at preventing recipients from developing genital warts or cervical cancer [4]. However, not all people get the vaccine, and many of those who are given the vaccine fail to receive the full vaccination schedule [5]. Further, the vaccine is only a preventative measure, so once an individual is infected by a high-risk HPV type, it cannot treat their infection [6]. Thus, a different method of intervention is needed once infection occurs and cervical cells become precancerous.

Currently, a surgical procedure called the Loop Electrosurgical Excision Procedure (LEEP) is the predominant treatment method, and in the United States, approximately 500,000 of these procedures are performed annually. But this procedure requires hospitalization and is associated with potential side effects, including interference with fertility. A non-surgical, patient-administered therapy would greatly improve the treatment of cervical precancer worldwide.

To this end, in vitro experiments showed that an FDA-approved, antimalarial derivative, artesunate, was able to selectively kill HPV 16- and HPV 18-positive cells [7]. In addition, a very recent published clinical trials demonstrated that artesunate can be self-administered by patients and can cure 70% of HPV precancers [8]. However, it would further improve the efficacy of this self-treatment procedure if the cure rate could approach 90%.

### SUMMARY

The present invention relates to a new combination therapy as a treatment for conditions and diseases associated with HPV. In particular, the combination therapy comprises one or more artemisinin-related compounds and one or more histone deacetylase (HDAC) inhibitors.

Some embodiments of the present invention relate to a method of treating an HPV-induced condition in a subject in need thereof, in which the method comprises administering one or more artemisinin-related compounds and one or more HDAC inhibitors to the subject. Optionally, the HPV-induced condition may be selected from the group consisting of cervical cancer, cervical dysplasia, vaginal cancer, vaginal dysplasia, vaginal papillomas, vulvar cancer, vulvar dysplasia, vulvar papillomas, anal cancer, anal dysplasia, anal papillomas, perianal cancer, perianal dysplasia, perianal papillomas, penile cancers, penile dysplasia, penile papillomas, oral cancer, oral dysplasia, oropharyngeal cancer, oropharyngeal dysplasia, oropharyngeal papillomas, laryngeal cancer, laryngeal dysplasia, laryngeal papillomas, sinonasal (nasal and paranasal sinuses) cancers, sinonasal dysplasia, and sinonasal papillomas.

Some embodiments of the present invention relate to a method of treating an HPV-induced lesion in a subject in need thereof, in which the method comprises administering one or more artemisinin-related compounds and one or more HDAC inhibitors to the subject. The HPV-induced condition may be benign, premalignant or malignant, and may be at a genital site such as the vagina, vulva, or penis; at a cervical site; at an anal site such as the rectum, anus, or perianal tissue; or at a site associated with the oral cavity.

Some embodiments of the present invention relate to a method of treating HPV-infected cells in a subject in need thereof, in which the method comprises administering one or more artemisinin-related compounds and one or more HDAC inhibitors to the subject. The HPV-infected cells may be benign, premalignant or malignant, and may be selected from cervical cells, vaginal cells, vulvar cells, penile cells, anal cells, rectal cells, perianal cells, oral cells, oropharyngeal cells, laryngeal cells, and sinonasal cells.

The one or more artemisinin-related compounds may be artemisinin, dihydroartemisinin, artemether, arteether, artesunate, artelinic acid, dihydroartemisinin propyl carbonate, or any combination thereof. In some embodiments, the one or more artemisinin-related compounds may be artemisinin or artesunate.

The one or more HDAC inhibitor may be trichostatin A, vorinostat, panobinostat, belinostat, givinostat, practinostat, quisinostat, abexinostat, CHR-3996, and AR-42, valproate, butyrate, entinostat, entinostat polymorph B, mocetinostat, chidamide, romidepsin, trapoxin, LAQ824, nicotinamide, cambinol, tenovin 1, tenovin 6, sirtinol, EX-527, tacedinaline, resminostat, HBI-8000, kevetrin, CUDC-101, tefinostat, 4SC202, rocilinostat, ME-344, or combinations thereof. In some embodiments, the one or more HDAC inhibitors may be panobinostat or vorinostat.

The one or more artemisinin-related compound and the one or more HDAC inhibitors each may be administered to the human subject by a route of delivery selected from oral administration, topical administration, parenteral administration, intravaginal administration, rectal administration, systemic administration, intramuscular administration, and intravenous administration. In some embodiments, the one or more artemisinin-related compound and the one or more HDAC inhibitors each may be administered to the human subject by oral or topical administration. In some embodiments, the one or more artemisinin-related compound and the one or more HDAC inhibitors may be administered to the human subject by the same route of delivery. In some embodiments, the one or more artemisinin-related compound and the one or more HDAC inhibitors may be administered to the human subject by different routes of delivery.

Additional aspects of the present invention relate to methods and treatment regimens comprising (a) administering to the subject one or more artemisinin-related compounds; and (b) administering to the subject one or more HDAC inhibitors. In some embodiments, the administration of the one or more artemisinin-related compounds is concurrent with the administration of the one or more HDAC inhibitors. In some embodiments, the administration of the one or more artemisinin-related compounds is before the administration of the one or more HDAC inhibitors. In some embodiments, the administration of the one or more artemisinin-related compounds is after the administration of the one or more HDAC inhibitors.

Further aspects of the invention relate to kits comprising pharmaceutical compositions of one or more artemisinin-related compounds and one or more HDAC inhibitors, and a package insert.

### BRIEF DESCRIPTION OF THE FIGURES

FIG. 1 shows dose-effect logarithmic curves of the percentage of cell viability after the respective treatment of each drug (panobinostat or artesunate) in each of the three HPV-positive cell lines (Caski, HeLa, and SiHa). Analyses were conducted using GraphPad Prism 7.

FIGS. 2A and 2B show the effect of artesunate and panobinostat individually and in combination. FIG. 2A shows a dose-effect curve depicting the relative dose of artesunate (ART), panobinostat (PAN), or the combination (A & P) based on the fraction of HeLa cells still surviving (Fa) after that treatment. FIG. 2B shows a combination index (CI) plot depicting the fraction of HeLa cells still surviving after the combination treatments of artesunate and panobinostat (Fa) based on the CI. The horizontal line at y=1 differentiates whether the relationship between the tested drugs is antagonistic (above the line), additive (on the line), or synergistic (under the line). All analyses were conducted using CompuSyn.

FIGS. 3A, 3B and 3C show dose-effect logarithmic curves of the percentage of cell viability after the respective treatment of each drug (panobinostat or artesunate) and the combination in each of the three cell lines (HeLa, SiHa and HEC/16E6E7). Analyses were conducted using GraphPad Prism 9.

FIGS. 4A, 4B and 4C show dose-effect logarithmic curves of the percentage of cell viability after the respective treatment of each drug (vorinostat or artesunate) and the combination in each of the three cell lines (HeLa, SiHa and HEC/16E6E7). Analyses were conducted using GraphPad Prism 9.

FIGS. 5A, 5B, and 5C shows a combination index (CI) plots depicting the fraction of HeLa cells (5A), Siha cells (5B), and HEC/16E6E7 cells (5C) still surviving after the combination treatments of artesunate (A) and panobinostat (P) based on the CI. The horizontal line at y=1 differentiates whether the relationship between the tested drugs is antagonistic (above the line), additive (on the line), or synergistic (under the line). All analyses were conducted using CompuSyn.

FIGS. 6A, 6B, and 6C shows a combination index (CI) plots depicting the fraction of HeLa cells (6A), Siha cells (6B), and HEC/16E6E7 cells (6C) still surviving after the combination treatments of artesunate (A) and vorinostat (S) based on the CI. The horizontal line at y=1 differentiates whether the relationship between the tested drugs is antagonistic (above the line), additive (on the line), or synergistic (under the line). All analyses were conducted using CompuSyn.

### DETAILED DESCRIPTION

The present invention relates to methods comprising the administration of one or more artemisinin-related compounds and one or more HDAC inhibitors; treatment regimens involving administration of one or more artemisinin-related compounds and one or more HDAC inhibitors; and kits comprising a pharmaceutical composition of an artemisinin-related compound, a pharmaceutical composition of an HDAC inhibitor, and a package insert.

The present invention is based, in part, on the unexpected discovery that the use of an artemisinin-related compound and an HDAC inhibitor in combination is effective in killing cells that are transformed by human papillomavirus, and that the combination yields a synergistic effect. Therefore, artemisinin-related compounds and HDAC inhibitors can be used in combination to treat HPV-induced conditions, including precancerous conditions, and to treat HPV-induced lesions.

Without wishing to be bound by any theory, it is believed the HDAC inhibitor is synergizing with the artemisinin-related compound by a mechanism independent of the artemisinin-related compound activity. For example, artemisinin-related compounds can kill cells by reaction with iron to generate toxic reactive oxygen species. The HDAC inhibitors are believed to be working, for example, to induce cell differentiation (and thereby inhibit conversion to cancer), inhibit HPV replication, increase recognition of HPV precancer cells by immune cells, increase exposure of E6/E7 antigens on the precancer cells, and/or induce apoptosis by iron-independent mechanisms.

### Artemisinin-Related Compounds

The term “artemisinin-related compound,” as used herein, refers to both artemisinin and artemisinin derivatives or analogs. Artemisinin (Qinghaosu) is a naturally occurring substance, obtained by purification from sweet wormwood, Artemisia annua. L. Artemisinin and its analogs are sesquiterpene lactones with a peroxide bridge.

For the present invention, artemisinin derivatives or analogs include, but are not limited to, dihydroartemisinin, artemether, artesunate, arteether, propylcarbonate dihydroartemisinin, and artelinic acid.

Other artemisinin derivatives or analogs for use in the present invention include, but are not limited to, artemisinin derivatives as described in U.S. patent application Ser. No. 10/545,356, which was granted as U.S. Pat. No. 7,989,491; U.S. patent application Ser. No. 16/496,743, which was published as U.S. Patent Publication No. 2020/0030284; U.S. patent application Ser. No. 16/099,195, which was published as U.S. Patent Publication No. 2019/0133997; U.S. patent application Ser. No. 15/531,241, which was published as U.S. Patent Publication No. 2017/0326102; U.S. patent application Ser. No. 14/125,032, which was granted as U.S. Pat. No. 9,623,005; U.S. patent application Ser. No. 15/303,170, which was granted as U.S. Pat. No. 9,999,621; U.S. patent application Ser. No. 14/757,433, which was granted as U.S. Pat. No. 9,918,972; U.S. patent application Ser. No. 14/904,671, which was granted as U.S. Pat. No. 9,802,952; U.S. patent application Ser. No. 14/651,298, which was granted as U.S. Pat. No. 9,603,831; and U.S. patent application Ser. No. 13/982,684, which was published as U.S. Patent Publication No. 2013/0317095; each of which is incorporated by reference herein.

The very low toxicity of these compounds to humans is a major benefit. Artesunate, for example, is twice as safe as artemether and only one-fiftieth as toxic as chloroquinine, the most common antimalarial drug.

### HDAC Inhibitors

The term “histone deacetylase” or “HDAC” refers to enzymes that remove the acetyl groups from the lysine residues in core histones, which may lead to the formation of a condensed and transcriptionally silenced chromatin. There are currently 18 known histone deacetylases, which are classified into four groups according to their homology to yeast HDACs: Class I HDACs, which include HDAC1, HDAC2, HDAC3, and HDAC8, and are related to the yeast RPD3 gene; Class II HDACs, which include HDAC4, HDACS, HDAC6, HDAC7, HDAC9, and HDAC10, and are related to the yeast Hdal gene; Class III HDACs, which are also known as the sirtuins and are related to the Sir2 gene; and Class IV HDAC, which is only HDAC11 and has features of both Class I and II HDACs. The term “histone deacetylase” or “HDAC” refers to any one or more of the 18 known histone deacetylases, unless otherwise specified. The term “histone deacetylase inhibitor” or “HDAC inhibitor,” as used herein, refers to a compound that selectively targets, decreases, or inhibits at least one activity of a histone deacetylase.

HDAC inhibitors according to the present invention include, but are not limited to, hydroxamic acid derivatives such as trichostatin A, vorinostat, panobinostat, belinostat, givinostat, practinostat, quisinostat, abexinostat, CHR-3996, and AR-42; carboxylic acid derivatives such as valproate and butyrate; benzamide derivatives such as entinostat, entinostat polymorph B, mocetinostat, and chidamide; cyclic peptides such as romidepsin; and epoxyketones such as trapoxins. Other HDAC inhibitors include LAQ824, nicotinamide, cambinol, tenovin 1, tenovin 6, sirtinol, EX-527, tacedinaline, resminostat, HBI-8000, kevetrin, CUDC-101, tefinostat, 4SC202, rocilinostat, and ME-344.

Other HDAC inhibitors for use in the present invention include, but are not limited to, HDAC inhibitors as described in U.S. patent application Ser. No. 12/093,069, which was granted as U.S. Pat. No. 8,828,392; U.S. patent application Ser. No. 15/558,370, which was granted as U.S. Pat. No. 10,532,053; U.S. patent application Ser. No. 14/907,321, which was granted as U.S. Pat. No. 9,751,832; U.S. patent application Ser. No. 15/592,929, which was granted as U.S. Pat. No. 10,385,131; U.S. patent application Ser. No. 15/124,246, which was granted as U.S. Pat. No. 10,213,422; and U.S. patent application Ser. No. 15/034,276, which was granted as U.S. Pat. No. 9,988,343; each of which is incorporated by reference herein.

In some embodiments, the HDAC inhibitor for the present invention may be selected from vorinostat, romidepsin, belinostat, panobinostat, and a combination thereof. In certain embodiments, the HDAC inhibitor may be panobinostat.

Importantly, HDAC inhibitors have been studied as a treatment for cancer, as their cell-intrinsic effects include induction of apoptosis and/or inhibition of cell proliferation [9]. HDAC inhibitors have been shown to exhibit immunostimulatory effects during cancer treatment [9, 10]. For example, HDAC inhibitors can sensitize tumor cells to immunotherapy by increasing tumor antigen expression in target tumor cells, as well as improve the anti-tumor activity of tumor antigen-specific lymphocytes [11, 12].

### Pharmaceutical Compositions

An aspect of the present invention relates to compositions comprising an active ingredient and one or more pharmaceutically acceptable excipients. The active ingredient may be one or more artemisinin-related compounds, one or more HDAC inhibitors, or both one or more artemisinin-related compounds and one or more HDAC inhibitors.

Compositions of the present invention include those suitable for oral/nasal, topical, parenteral, intravaginal and/or rectal administration. The compositions may conveniently be presented in a unit dosage form and may be prepared by any methods well known in the art of pharmacy. The amount of active ingredient which can be combined with a carrier material to produce a single dosage form will vary depending upon the host being treated and the particular route of administration. The amount of active ingredient which can be combined with a carrier material to produce a single dosage form will generally be that amount of the compound which produces a therapeutic effect.

Compositions of the present invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the active ingredient. An artemisinin-related compound may also be administered as a bolus, electuary or paste.

In solid dosage forms for oral administration (e.g., capsules, tablets, pills, dragees, powders, granules, and the like, including for use in foods such as gum, gummy candy, as examples), the active ingredient may be combined with one or more pharmaceutically acceptable carriers, such as sodium citrate or dicalcium phosphate, and/or any of the following: (a) fillers or extenders, such as starches, lactose, sucrose, glucose, mannitol, silicic acid, or mixtures thereof; (b) binders, such as, for example, alginates, gelatin, acacia, sucrose, various celluloses, cross-linked polyvinylpyrrolidone, microcrystalline cellulose (e.g., AVICEL® PH-101, AVICEL® PH-102), silicified microcrystalline cellulose (e.g., PROSOLV® SMCC), carboxymethylcellulose, or mixtures thereof; (c) humectants, such as glycerol; (d) disintegrating agents, such as agar-agar, calcium carbonate, alginic acid, certain silicates, sodium carbonate, sodium starch glycolate, lightly crosslinked polyvinyl pyrrolidone, corn starch, potato starch, maize starch, croscarmellose sodium, cross-povidone, or mixtures thereof; (e) solution retarding agents, such as paraffin; (f) absorption accelerators, such as quaternary ammonium compounds; (g) wetting agents, such as, for example, cetyl alcohol, glycerol monostearate, or poloxamers such as poloxamer 407 (e.g., PLURONIC® F.-127) or poloxamer 188 (e.g., PLURONIC® F-68), or mixtures thereof; (h) absorbents, such as kaolin and bentonite clay; (i) lubricants, such a talc, calcium stearate, magnesium stearate, solid polyethylene glycols, sodium lauryl sulfate, colloidal silicon dioxide (i.e., hydrophobic colloidal silica, such as AEROSIL®, stearic acid, silica gel, or mixtures thereof; and (j) coloring agents. In the case of capsules, tablets and pills, the pharmaceutical compositions may also comprise a buffering agent, such as, but not limited to, triethylamine, meglumine, diethanolamine, ammonium acetate, arginine, lysine, histidine, a phosphate buffer (e.g., sodium phosphate tribasic, sodium phosphate dibasic, sodium phosphate monobasic, or o-phosphoric acid), sodium bicarbonate, a Britton-Robinson buffer, a Tris buffer (containing Tris(hydroxymethyl)aminomethane), a HEPES buffer (containing N-(2-hydroxyethyl)piperazine-N′-(2-ethanesulfonic acid), acetate, a citrate buffer (e.g., citric acid, citric acid anhydrous, citrate monobasic, citrate dibasic, citrate tribasic, citrate salt), ascorbate, glycine, glutamate, lactate, malate, formate, sulfate, and mixtures thereof. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the artemisinin-related compounds, HDAC inhibitors, or a combination thereof, include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents including those listed herein, emulsifying and suspending agents, sweetening, flavoring, coloring, perfuming, and preservative agents.

Suspensions, in addition to the active ingredients, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In particular, methods of the invention can be administered topically, either to skin or to mucosal membranes such as those on the cervix and vagina. The topical formulations may comprise the excipients described for the solid and liquid composition set forth above, and may further include one or more of the wide variety of agents known to be effective as skin or stratum corneum penetration enhancers. Examples of such agents include 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art, e.g., salicylic acid and sulfur, may also be included.

Dosage forms for the topical or transdermal administration of an active ingredient may include powders, sprays, ointments, pastes, creams, lotions, gels, solutions, patches, and inhalants. The active ingredient may be mixed under sterile conditions with a pharmaceutically acceptable carrier, and with any preservatives, buffers, or propellants which may be required. The ointments, pastes, creams and gels may contain, in addition to the active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise an active ingredient in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of antioxidants that that may be used in the pharmaceutical compositions of the present invention include, but are not limited to, acetylcysteine, ascorbyl palmitate, butylated hydroxyanisole, butylated hydroxytoluene, monothioglycerol, potassium nitrate, sodium ascorbate, sodium formaldehyde sulfoxylate, sodium metabisulfite, sodium bisulfite, vitamin E or a derivative thereof, propyl gallate, edetate (e.g., disodium edetate), diethylenetriaminepentaacetic acid, bismuth sodium triglycollamate, or a combination thereof. Antioxidants may also comprise amino acids such as methionine, histidine, cysteine and those carrying a charged side chain, such as arginine, lysine, aspartic acid, and glutamic acid. Any stereoisomer (e.g., l-, d-, or a combination thereof) of any particular amino acid (e.g., methionine, histidine, arginine, lysine, isoleucine, aspartic acid, tryptophan, threonine and combinations thereof) or combinations of these stereoisomers, may be present so long as the amino acid is present either in its free base form or its salt form.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Surfactants that that may be used in the pharmaceutical compositions of the present invention may include, but are not limited to, sodium lauryl sulfate, dioctyl sodium sulfosuccinate, dioctyl sodium sulfonate, benzalkonium chloride, benzethonium chloride, lauromacrogol 400, polyoxyl 40 stearate, polyoxyethylene hydrogenated castor oil (e.g., polyoxyethylene hydrogenated castor oil 10, 50, or 60), glycerol monostearate, polysorbate (e.g., polysorbate 40, 60, 65 or 80), sucrose fatty acid ester, methyl cellulose, polyalcohols and ethoxylated polyalcohols, thiols (e.g., mercaptans) and derivatives, poloxamers, polyethylene glycol-fatty acid esters (e.g., KOLLIPHOR® RH40, KOLLIPHOR® EL), lecithins, and mixtures thereof.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of the active ingredient in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Compositions of the active ingredient for intravaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Optionally, such compositions suitable for vaginal administration also include pessaries, tampons, creams, gels, pastes, foams or spray formulations containing such carriers as are known in the art to be appropriate. In some embodiments, the compositions may be suitable for use with devices such as vaginal or cervical rings.

Compositions of the present invention, including those used for oral/nasal, topical, parenteral, intravaginal and/or rectal administration may further comprise one or more pH-adjusting agents. Such pH-adjusting agents include pharmaceutically acceptable acids or bases. For example, acids may include, but are not limited to, one or more inorganic mineral acids such as hydrochloric, hydrobromic, sulfuric, phosphoric, nitric, and the like; or one or more organic acids such as acetic, succinic, tartaric, ascorbic, citric, glutamic, benzoic, methanesulfonic, ethanesulfonic, trifluoroacetic, and the like. Bases may be one or more inorganic bases or organic bases, including, but not limited to, alkaline carbonate, alkaline bicarbonate, alkaline earth metal carbonate, alkaline hydroxide, alkaline earth metal hydroxide, or amine. For example, the inorganic or organic base may be an alkaline hydroxide such as lithium hydroxide, potassium hydroxide, cesium hydroxide, sodium hydroxide, or the like; an alkaline carbonate such as calcium carbonate, sodium carbonate, or the like; or an alkaline bicarbonate such as sodium bicarbonate, or the like; the organic base may also be sodium acetate.

In embodiments in which the pharmaceutical compositions comprises both one or more artemisinin-related compounds and one or more HDAC inhibitors, the one or more pharmaceutically acceptable excipients should be compatible with both the one or more artemisinin-related compounds and the one or more HDAC inhibitors. In some embodiments, “compatible” in this context may mean that the one or more pharmaceutically acceptable excipients do not negatively impact one or more properties of the one or more artemisinin-related compounds and the one or more HDAC inhibitors, such as to reduce the stability or efficacy of the one or more artemisinin-related compounds and the one or more HDAC inhibitors. In some embodiments, “compatible” in this context may also mean, or may alternatively mean, that the one or more pharmaceutically acceptable excipients can achieve their intended function in the presence of the one or more artemisinin-related compounds and the one or more HDAC inhibitors; for example, a compatible solvent is capable of dissolving both the one or more artemisinin-related compounds and the one or more HDAC inhibitors, a compatible antioxidant functions as or maintains its properties of an antioxidant in the presence of both the one or more artemisinin-related compounds and the one or more HDAC inhibitors, etc.

In embodiments of the invention, the pharmaceutical composition comprising the one or more artemisinin-related compounds and the pharmaceutical composition comprising the one or more HDAC inhibitors may be for different routes of delivery. For instance, the pharmaceutical composition comprising the one or more artemisinin-related compounds may be for oral delivery while the pharmaceutical composition comprising the one or more HDAC inhibitors may be for intravenous delivery; or vice versa. Optionally, the pharmaceutical composition comprising the one or more artemisinin-related compounds may be for topical delivery while the pharmaceutical composition comprising the one or more HDAC inhibitors may be for subcutaneous delivery; or vice versa. Alternatively, the pharmaceutical composition comprising the one or more artemisinin-related compounds and the pharmaceutical composition comprising the one or more HDAC inhibitors may be for the same route of delivery.

In certain embodiments, the active ingredient can be administered to animals in animal feed. For example, these compounds can be included in an appropriate feed premix, which is then incorporated into the complete ration in a quantity sufficient to provide a therapeutically effective amount to the animal. Alternatively, an intermediate concentrate or feed supplement containing the artemisinin-related compounds can be blended into the feed. The way in which such feed premixes and complete rations can be prepared and administered are described in reference books (see, e.g., “Applied Animal Nutrition,” W. H. Freedman and CO., San Francisco, U.S.A., 1969 or “Livestock Feeds and Feeding,” 0 and B books, Corvallis, Ore., U.S.A., 1977).

The pharmaceutical compositions of the present invention may be prepared using methods known in the art. For example, the active ingredient and the one or more pharmaceutically acceptable excipients may be mixed by simple mixing, or may be mixed with a mixing device continuously, periodically, or a combination thereof. Examples of mixing devices may include, but are not limited to, a magnetic stirrer, shaker, a paddle mixer, homogenizer, and any combination thereof.

### Treatments Using Artemisinin-Related Compounds and HDAC Inhibitors

An aspect of the present invention relates to the use of artemisinin-related compounds and HDAC inhibitors to treat an HPV-induced condition. Some embodiments relate to methods of treating an HPV-induced condition in a subject in need thereof, the method comprising administering one or more artemisinin-related compounds and one or more HDAC inhibitors to the subject. Some embodiments relate to the use of one or more artemisinin-related compounds and one or more HDAC inhibitors for treating an HPV-induced condition in a subject in need thereof, the use comprising administering the one or more artemisinin-related compounds and the one or more HDAC inhibitors to the subject. Some embodiments relate to one or more artemisinin-related compounds and one or more HDAC inhibitors of the present invention for use in treating an HPV-induced condition in a subject in need thereof, the use comprising administering the one or more artemisinin-related compounds and the one or more HDAC inhibitors to the subject. Some embodiments relate to a use of one or more artemisinin-related compounds and one or more HDAC inhibitors in the manufacture of a medicament for treating an HPV-induced condition in a subject in need thereof. Some embodiments relate to a treatment regimen for treating an HPV-induced condition in a subject in need thereof, the regimen comprising (a) administering to the subject one or more artemisinin-related compounds; and (b) administering to the subject one or more HDAC inhibitors.

The HPV-induced condition may be cervical cancer, cervical dysplasia, vaginal cancer, vaginal dysplasia, vaginal papillomas, vulvar cancer, vulvar dysplasia, vulvar papillomas, anal cancer, anal dysplasia, anal papillomas, perianal cancer, perianal dysplasia, perianal papillomas, penile cancers, penile dysplasia, penile papillomas, oral cancer, oral dysplasia, oropharyngeal cancer, oropharyngeal dysplasia, oropharyngeal papillomas, laryngeal cancer, laryngeal dysplasia, laryngeal papillomas, sinonasal (nasal and paranasal sinuses) cancers, sinonasal dysplasia, or sinonasal papillomas. In some embodiments, the HPV-induced condition may be cervical cancer or cervical dysplasia.

In some embodiments, treatment of an HPV-induced condition may be demonstrated by one or more of the following: (i) amelioration of one or more causes or symptoms of the condition; (ii) inhibition of one or more symptoms of the condition from worsening; (iii) elimination of one or more symptoms of the condition; (iv) elimination of the condition itself; (v) inhibition of formation of a tumor; (vi) reduction in the size of a tumor; (vii) inhibition in growth of a tumor; (viii) decrease in known biomarkers associated with the HPV-induced condition; (ix) prevention of increase of known biomarkers associated with the HPV-induced condition; (x) elimination of known biomarkers associated with the HPV-induced condition; (xi) inhibition or decrease of expression of HPV genes and/or proteins associated with viral replication or infection such as virus replication proteins E1, E2, E4, E5, E6, and/or E7; and virus structural proteins L1 and L2; and (xii) a combination thereof.

An aspect of the present invention also relates to the use of artemisinin-related compounds and HDAC inhibitors to treat an HPV-induced lesion. Some embodiments relate to methods of treating an HPV-induced lesion in a subject in need thereof, the method comprising administering one or more artemisinin-related compounds and one or more HDAC inhibitors to the subject. Some embodiments relate to the use of one or more artemisinin-related compounds and one or more HDAC inhibitors for treating an HPV-induced lesion in a subject in need thereof, the use comprising administering the one or more artemisinin-related compounds and the one or more HDAC inhibitors to the subject. Some embodiments relate to one or more artemisinin-related compounds and one or more HDAC inhibitors of the present invention for use in treating an HPV-induced lesion in a subject in need thereof, the use comprising administering the one or more artemisinin-related compounds and the one or more HDAC inhibitors to the subject. Some embodiments relate to a use of one or more artemisinin-related compounds and one or more HDAC inhibitors in the manufacture of a medicament for treating an HPV-induced lesion in a subject in need thereof. Some embodiments relate to a treatment regimen for treating an HPV-induced lesion in a subject in need thereof, the regimen comprising (a) administering to the subject one or more artemisinin-related compounds; and (b) administering to the subject one or more HDAC inhibitors.

The HPV-induced lesion may be benign, premalignant or malignant.

In some embodiments, the HPV-induced lesion may be a lesion of the cervix. In certain embodiments, the HPV-induced lesion may be a benign lesion of the cervix. In certain embodiments, the HPV-induced lesion may be a premalignant lesion of the cervix. In certain embodiments, the HPV-induced lesion may be a malignant lesion of the cervix.

In some embodiments, the HPV-induced lesion may be at a genital site. In certain embodiments, the HPV-induced lesion may be a benign lesion of the vagina. In certain embodiments, the HPV-induced lesion may be a premalignant lesion of the vagina. In certain embodiments, the HPV-induced lesion may be a malignant lesion of the vagina. In certain embodiments, the HPV-induced lesion may be a benign lesion of the vulva. In certain embodiments, the HPV-induced lesion may be a premalignant lesion of the vulva. In certain embodiments, the HPV-induced lesion may be a malignant lesion of the vulva. In certain embodiments, the HPV-induced lesion may be a benign lesion of the penis. In certain embodiments, the HPV-induced lesion may be a premalignant lesion of the penis. In certain embodiments, the HPV-induced lesion may be a malignant lesion of the penis.

In some embodiments, the HPV-induced lesion may be at an anal site. In certain embodiments, the HPV-induced lesion may be a benign lesion of the rectum. In certain embodiments, the HPV-induced lesion may be a premalignant lesion of the rectum. In certain embodiments, the HPV-induced lesion may be a malignant lesion of the rectum. In certain embodiments, the HPV-induced lesion may be a benign lesion of the anus. In certain embodiments, the HPV-induced lesion may be a premalignant lesion of the anus. In certain embodiments, the HPV-induced lesion may be a malignant lesion of the anus. In certain embodiments, the HPV-induced lesion may be a benign lesion of the perianal tissue. In certain embodiments, the HPV-induced lesion may be a premalignant lesion of the perianal tissue. In certain embodiments, the HPV-induced lesion may be a malignant lesion of the perianal tissue.

In some embodiments, treatment of an HPV-induced lesion may be demonstrated by one or more of the following: (i) amelioration of one or more causes or symptoms stemming from the lesion; (ii) inhibition of one or more symptoms stemming from the lesion from worsening; (iii) elimination of one or more symptoms stemming from the lesion; (iv) inhibition of growth of the lesion; (v) reduction of the size of the lesion; (vi) elimination of the lesion; (vii) decrease in known biomarkers associated with the HPV-induced lesion; (viii) prevention of increase of known biomarkers associated with the HPV-induced lesion; (ix) elimination of known biomarkers associated with the HPV-induced lesion; (x) inhibition or decrease of expression of HPV genes and/or proteins associated with viral replication or infection such as virus replication proteins E1, E2, E4, E5, E6, and/or E7; and virus structural proteins L1 and L2; and (xi) a combination thereof.

Further, an aspect of the present invention relates to the use of artemisinin-related compounds and HDAC inhibitors to treat HPV-infected cells in a subject in need thereof. Some embodiments relate to methods of treating HPV-infected cells of a subject in need thereof, the method comprising administering one or more artemisinin-related compounds and one or more HDAC inhibitors to the subject. Some embodiments relate to the use of one or more artemisinin-related compounds and one or more HDAC inhibitors for treating HPV-infected cells in a subject in need thereof, the use comprising administering the one or more artemisinin-related compounds and the one or more HDAC inhibitors to the subject. Some embodiments relate to one or more artemisinin-related compounds and one or more HDAC inhibitors of the present invention for use in treating HPV-infected cells in a subject in need thereof, the use comprising administering the one or more artemisinin-related compounds and the one or more HDAC inhibitors to the subject. Some embodiments relate to a use of one or more artemisinin-related compounds and one or more HDAC inhibitors in the manufacture of a medicament for treating HPV-infected cells lesion in a subject in need thereof. Some embodiments relate to a regimen for treating HPV-infected cells in a subject in need thereof, the regimen comprising (a) administering to the subject one or more artemisinin-related compounds; and (b) administering to the subject one or more HDAC inhibitors.

The HPV-infected cells may be benign, premalignant or malignant. In some embodiments, the HPV-infected cells may be benign cervical cells. In some embodiments, the HPV-infected cells may be premalignant cervical cells. In some embodiments, the HPV-infected cells may be malignant cervical cells. In some embodiments, the HPV-infected cells may be benign vaginal cells. In some embodiments, the HPV-infected cells may be premalignant vaginal cells. In some embodiments, the HPV-infected cells may be malignant vaginal cells. In some embodiments, the HPV-infected cells may be benign vulvar cells. In some embodiments, the HPV-infected cells may be premalignant vulvar cells. In some embodiments, the HPV-infected cells may be malignant vulvar cells. In some embodiments, the HPV-infected cells may be benign penile cells. In some embodiments, the HPV-infected cells may be premalignant penile cells. In some embodiments, the HPV-infected cells may be malignant penile cells. In some embodiments, the HPV-infected cells may be benign anal cells. In some embodiments, the HPV-infected cells may be premalignant anal cells. In some embodiments, the HPV-infected cells may be malignant anal cells. In some embodiments, the HPV-infected cells may be benign rectal cells. In some embodiments, the HPV-infected cells may be premalignant rectal cells. In some embodiments, the HPV-infected cells may be malignant rectal cells. In some embodiments, the HPV-infected cells may be benign perianal cells. In some embodiments, the HPV-infected cells may be premalignant perianal cells. In some embodiments, the HPV-infected cells may be malignant perianal cells.

In some embodiments, treatment of an HPV-infected cells may be demonstrated by one or more of the following: (i) amelioration of one or more causes or symptoms of the HPV-infected cells; (ii) inhibition of one or more symptoms of the infected cells from worsening; (iii) elimination of one or more symptoms of the HPV-infected cells; (iv) inducing apoptosis of the HPV-infected cells, or a portion of the HPV-infected cells; (v) inhibition of growth of the HPV-infected cells, or a portion of the HPV-infected cells; (vi) decrease in known biomarkers associated with the HPV-infected cells; (vii) prevention of increase of known biomarkers associated with the HPV-infected cells; (viii) elimination of known biomarkers associated with the HPV-infected cells; (ix) inhibition or decrease of expression of HPV genes and/or proteins associated with viral replication or infection such as virus replication proteins E1, E2, E4, E5, E6, and/or E7; and virus structural proteins L1 and L2; and (x) a combination thereof.

The subject in the methods of the present invention may be a mammal, which includes, but is not limited to, a human, monkey, cow, hog, sheep, horse, dog, cat, rabbit, rat, and mouse. In certain embodiments, the subject is a human. In particular embodiments, the subject is a human patient.

In certain embodiments, the one or more artemisinin-related compounds are administered to the subject in a therapeutically effective amount. The phrase “therapeutically effective amount,” as used in the context of the artemisinin-related compounds herein, may in some embodiments refer to a quantity sufficient to elicit the biological or medical response that is being sought, including treatment of an HPV-induced condition, treatment of an HPV-induced lesion, or treating HPV-infected cells.

Artemisinin is a relatively safe drug and produces few side-effects, even at high doses. Oral doses of 70 mg/kg/day for six days have been used in humans for malaria treatment. Furthermore, more potent analogs of this and similar compounds are also available. Higher efficacy of artemisinin action can be achieved by other means. For example, artemisinin is more reactive with heme than with free iron [13]. Iron can be introduced into target cells using transferrin [14] or the heme-carrying compound hemoplexin [15, 16]. The concentrations of agents for enhancing intracellular iron concentrations in the practice of the present invention will generally range up to the maximally tolerated dose for a particular subject and agent, which will vary depending on the agent, subject, disease condition and other factors. Dosages ranging from about 1 to about 100 mg of iron per kilogram of subject body weight per day will generally be useful for this purpose.

Dosage levels of the artemisinin-related compounds may be varied so as to obtain amounts at the site of target cells (e.g., virus infected cells or abnormal cervical cells), effective to obtain the desired therapeutic or prophylactic response. Accordingly, the therapeutically effective amount of artemisinin-related compounds will depend on the nature and site of the target cells, the desired quantity of artemisinin-related compounds required at the target cells for inhibition or killing, the nature of the artemisinin-related compounds employed, the route of administration, the physical condition and body size of the subject, and other factors.

A therapeutically effective amount of artemisinin-related compounds may be presented as different units. For example, a therapeutically effective amount of artemisinin-related compounds may be presented as a fixed dose. Thus, in some embodiments, a therapeutically effective amount of artemisinin-related compounds may be about 0.1 mg to about 500 mg, or about 0.1 mg to about 400 mg, or about 0.1 mg to about 300 mg, or about 1 mg to about 200 mg, or about 1 mg to about 100 mg; or any amount therebetween, such as about 1 mg, or about 5 mg, or about 10 mg, or about 20 mg, or about 30 mg, or about 40 mg, or about 50 mg, or about 60 mg, or about 70 mg, or about 80 mg, or about 90 mg, or about 100 mg, or about 120 mg, or about 140 mg, or about 160 mg, or about 180 mg, or about 200 mg, or about 220 mg, or about 240 mg, or about 260 mg, or about 280 mg, or about 300 mg, or about 320 mg, or about 340 mg, or about 360 mg, or about 380 mg, or about 400 mg, or about 420 mg, or about 440 mg, or about 460 mg, or about 480 mg, or about 500 mg.

A therapeutically effective amount of artemisinin-related compounds may also be presented in units of weight of artemisinin-related compounds per body weight of the subject. Thus, in some embodiments, a therapeutically effective amount of artemisinin-related compounds may be about 0.1 mg to about 500 mg per kilogram of body weight (i.e., about 0.1 mg/kg to about 500 mg/kg), or about 0.1 mg/kg to about 400 mg/kg, or about 0.1 mg/kg to about 300 mg/kg, or about 1 mg/kg to about 200 mg/kg, or about 1 mg/kg to about 100 mg/kg; or any amount therebetween, such as about 1 mg/kg, or about 5 mg/kg, or about 10 mg/kg, or about 20 mg/kg, or about 30 mg/kg, or about 40 mg/kg, or about 50 mg/kg, or about 60 mg/kg, or about 70 mg/kg, or about 80 mg/kg, or about 90 mg/kg, or about 100 mg/kg, or about 120 mg/kg, or about 140 mg/kg, or about 160 mg/kg, or about 180 mg/kg, or about 200 mg/kg, or about 220 mg/kg, or about 240 mg/kg, or about 260 mg/kg, or about 280 mg/kg, or about 300 mg/kg, or about 320 mg/kg, or about 340 mg/kg, or about 360 mg/kg, or about 380 mg/kg, or about 400 mg/kg, or about 420 mg/k, or about 440 mg/k or about 460 mg/kg, or about 480 mg/kg, or about 500 mg/kg.

Further, a therapeutically effective amount of the artemisinin-related compounds may be presented in units of weight of the artemisinin-related compounds per body area of the subject. Thus, in some embodiments, a therapeutically effective amount of artemisinin-related compounds may be about 0.1 mg to about 2000 mg per square meter of the subject's body area (i.e., about 0.1 mg/m² to about 2000 mg/m²), or about 0.1 mg/m² to about 1900 mg/m², or about 0.1 mg/m² to about 1800 mg/m², or about 0.1 mg/m² to about 1700 mg/m², or about 0.1 mg/m² to about 1600 mg/m², or about 1 mg/m² to about 1500 mg/m², or about 1 mg/m² to about 1400 mg/m², or about 1 mg/m² to about 1300 mg/m², or about 1 mg/m² to about 1200 mg/m², or about 1 mg/m² to about 1100 mg/m², or about 5 mg/m² to about 1000 mg/m², or about 5 mg/m² to about 900 mg/m², or about 5 mg/m² to about 800 mg/m², or about 10 mg/m² to about 700 mg/m², or about 10 mg/m² to about 600 mg/m², or about 10 mg/m² to about 500 mg/m²; or any amount therebetween, such as about 1 mg/m², or about 5 mg/m², or about 10 mg/m², or about 15 mg/m², or about 20 mg/m², or about 30 mg/m², or about 40 mg/m², or about 50 mg/m², or about 60 mg/m², or about 70 mg/m², or about 80 mg/m², or about 90 mg/m², or about 100 mg/m², or about 120 mg/m², or about 140 mg/m², or about 160 mg/m², or about 180 mg/m², or about 200 mg/m², or about 220 mg/m², or about 240 mg/m², or about 260 mg/m², or about 280 mg/m², or about 300 mg/m², or about 320 mg/m², or about 340 mg/m², or about 360 mg/m², or about 380 mg/m², or about 400 mg/m², or about 420 mg/m², or about 440 mg/m², or about 460 mg/m², or about 480 mg/m², or about 500 mg/m², or about 550 mg/m², or about 600 mg/m², or about 650 mg/m², or about 700 mg/m², or about 750 mg/m², or about 800 mg/m², or about 850 mg/m², or about 900 mg/m², or about 1000 mg/m², or about 1050 mg/m², or about 1100 mg/m², or about 1150 mg/m², or about 1200 mg/m², or about 1250 mg/m², or about 1300 mg/m², or about 1350 mg/m², or about 1400 mg/m², or about 1450 mg/m², or about 1500 mg/m², or about 1550 mg/m², or about 1600 mg/m², or about 1650 mg/m², or about 1700 mg/m², or about 1750 mg/m², or about 1800 mg/m², or about 1850 mg/m², or about 1900 mg/m², or about 1950 mg/m², or about 2000 mg/m².

In embodiments, the one or more HDAC inhibitors are administered to the subject in a therapeutically effective amount. The phrase “therapeutically effective amount,” as used in the context of the HDAC inhibitors herein may in some embodiments refer to a quantity sufficient to elicit the biological or medical response that is being sought, including treatment of an HPV-induced condition, treatment of an HPV-induced lesion, or treating HPV-infected cells.

Dosage levels of the HDAC inhibitor may be varied so as to obtain amounts at the site of target cells (e.g., virus infected cells or abnormal cervical cells), effective to obtain the desired therapeutic or prophylactic response. Accordingly, the therapeutically effective amount of HDAC inhibitor will depend on the nature and site of the target cells, the desired quantity of HDAC inhibitor required at the target cells for inhibition or killing, the nature of the HDAC inhibitor employed, the route of administration, the physical condition and body size of the subject, and other factors.

A therapeutically effective amount of HDAC inhibitor may be presented as different units. For example, a therapeutically effective amount of HDAC inhibitor may presented as a fixed dose. Thus, in some embodiments, a therapeutically effective amount of HDAC inhibitor may be about 0.1 ng to about 500 mg, or about 1 ng to about 400 mg, or about 10 ng to about 300 mg, or about 100 ng to about 200 mg, or about 1000 ng to about 100 mg; or any amount therebetween, such as about 0.1 ng, or about 0.5 ng, or about 1 ng, or about 5 ng, or about 10 ng, or about 50 ng, or about 100 ng, or about 500 ng, or about 1000 ng, or about 5000 ng, or about 0.01 mg, or about 0.05 mg, or about 0.1 mg, or about 0.5 mg, or about 1 mg, or about 5 mg, or about 10 mg, or about 20 mg, or about 30 mg, or about 40 mg, or about 50 mg, or about 100 mg, or about 200 mg, or about 300 mg, or about 400 mg, or about 500 mg.

A therapeutically effective amount of HDAC inhibitor may also be presented in units of weight of the HDAC inhibitor per body weight of the subject. Thus, in some embodiments, a therapeutically effective amount of HDAC inhibitor may be about 0.1 ng to about 500 mg per kilogram of body weight (i.e., about 0.1 ng/kg to about 500 mg/kg), or about 1 ng/kg to about 400 mg/kg, or about 10 ng/kg to about 300 mg/kg, or about 100 ng/kg to about 200 mg/kg, or about 1000 ng/kg to about 100 mg/kg; or any amount therebetween, such as about 0.1 ng/kg, or about 0.5 ng/kg, or about 1 ng/kg, or about 5 ng/kg, or about 10 ng/kg, or about 50 ng/kg, or about 100 ng/kg, or about 500 ng/kg, or about 1000 ng/kg, or about 5000 ng/kg, or about 0.01 mg/kg, or about 0.05 mg/kg, or about 0.1 mg/kg, or about 0.5 mg/kg, or about 1 mg/kg, or about 5 mg/kg, or about 10 mg/kg, or about 50 mg/kg, or about 100 mg/kg, or about 200 mg/kg, or about 300 mg/kg, or about 400 mg/kg, or about 500 mg/kg.

Further, a therapeutically effective amount of HDAC inhibitor may be presented in units of weight of the HDAC inhibitor per body area of the subject. Thus, in some embodiments, a therapeutically effective amount of HDAC inhibitor may be about 0.1 ng to about 2000 mg per square meter of the subject's body area (i.e., about 0.1 ng/m² to about 2000 mg/m²), or about 0.5 ng/m² to about 1800 mg/m², or about 1 ng/m² to about 1600 mg/m², or about 5 ng/m² to about 1400 mg/m², or about 10 ng/m² to about 1200 mg/m², or about 50 ng/m² to about 1000 mg/m², or about 100 ng/m² to about 800 mg/m², or about 500 ng/m² to about 600 mg/m², or about 1000 ng/m² to about 500 mg/m²; or any amount therebetween, such as about 0.1 ng/m², or about 0.5 ng/m², or about 1 ng/m², or about 5 ng/m², or about 10 ng/m², or about 50 ng/m², or about 100 ng/m², or about 500 ng/m², or about 1000 ng/m², or about 5000 ng/m², or about 0.01 mg/m², or about 0.05 mg/m², or about 0.1 mg/m², or about 0.5 mg/m², or about 1 mg/m², or about 5 mg/m², or about 10 mg/m², or about 50 mg/m², or about 100 mg/m², or about 200 mg/m², or about 300 mg/m², or about 400 mg/m², or about 500 mg/m², or about 1000 mg/m², or about 1500 mg/m², or about 2000 mg/m².

In some embodiments, the one or more artemisinin-related compounds may be administered concurrently with the administration of the one or more HDAC inhibitors. The term “concurrently” or “concomitantly” (or other forms of these words such as “concurrent” or “concomitant,” respectively) as used herein may mean that the one or more artemisinin-related compounds is administered to the subject within about 15 minutes or less, or within about 10 minutes or less, or within about 5 minutes or less, or within about 4 minutes or less, or within about 3 minutes or less, or within about 2 minutes or less, or within about 1 minute or less, or simultaneously, of the administration of the one or more HDAC inhibitors.

In some embodiments, the one or more artemisinin-related compounds may be administered before the administration of the one or more HDAC inhibitors. In certain embodiments, the one or more artemisinin-related compounds may be administered shortly before the administration of the one or more HDAC inhibitors. The term “shortly before” as used herein may mean that the one or more artemisinin-related compounds is administered to the subject about 4 hours or less, or about 3 hours or less, or about 2 hours or less, or about 1 hour or less, or about 45 minutes or less, or about 30 minutes or less, or about 15 minutes or less, prior to the administration of the one or more HDAC inhibitors.

In some embodiments, the one or more artemisinin-related compounds may be administered after the administration of the one or more HDAC inhibitors. In certain embodiments, the one or more artemisinin-related compounds may be administered shortly after the administration of the one or more HDAC inhibitors. The term “shortly after” as used herein means that the one or more artemisinin-related compounds is administered to the subject about 4 hours or less, or about 3 hours or less, or about 2 hours or less, or about 1 hour or less, or about 45 minutes or less, or about 30 minutes or less, or about 15 minutes or less, after the administration of the one or more HDAC inhibitors.

In embodiments of the invention, the one or more artemisinin-related compounds and the one or more HDAC inhibitors may be administered all at once (once-daily dosing), or may be divided and administered more frequently (such as twice-per-day dosing). In some embodiments, the one or more artemisinin-related compounds and the one or more HDAC inhibitors may be administered every other day, or every three days, or every four days, or every five days, or every six days, or once per week, or once per two weeks, or once every three weeks, or once every four weeks, or once every five weeks, or once every six weeks, or once every seven weeks, or once every eight weeks, or once every two months, once every three months, once every four months, once every five months, once every six months, once every seven months, once every eight months, once every nine months, once every ten months, once every eleven months, once every twelve months, once every year, or periods of time therebetween. In some embodiments, the one or more artemisinin-related compounds and/or the one or more HDAC inhibitors may be administered as a loading dose followed by one or more maintenance doses.

In some embodiments, every administration of the one or more artemisinin-related compounds may not be accompanied by an administration of the one or more HDAC inhibitors, or vice versa. As an example, the one or more artemisinin-related compounds may be administered daily and the one or more HDAC inhibitors may be administered every other day. Optionally, the one or more HDAC inhibitors may be administered as a loading dose followed by bi-weekly maintenance doses, and the one or more artemisinin-related compounds may be administered daily.

In embodiments of the invention, administration of the one or more artemisinin-related compounds and the one or more HDAC inhibitors may be preceded by a step of identifying the subject in need thereof, i.e., identifying the subject having an HPV-induced condition, an HPV-induced lesion, or HPV-infected cells. Such identification of the subject may be achieved by methods known in the art for diagnosing the presence of cancer, cancerous lesions, precancerous lesions, precancerous cells, HPV-infected cells, etc., in the cervix, vagina, vulva, penis, rectum, anus, mouth, etc.

In embodiments of the invention, administration of the one or more artemisinin-related compounds and administration of the one or more HDAC inhibitors may have an additive effect. The term “additive effect” as used herein means that the effect of administering the combination of the one or more artemisinin-related compounds and the one or more HDAC inhibitors to, for example, treat an HPV-induced condition, treat an HPV-induced lesion, or treat HPV-infected cells, is approximately equal to the addition of the effect of administering the one or more artemisinin-related compounds by themselves and the effect of administering the one or more HDAC inhibitors by themselves.

In embodiments of the invention, administration of the one or more artemisinin-related compounds and administration of the one or more HDAC inhibitors may have a synergistic effect. The term “synergistic effect” as used herein means that the effect of administering the combination of the one or more artemisinin-related compounds and the one or more HDAC inhibitors to, for example, treat an HPV-induced condition, treat an HPV-induced lesion, or treat HPV-infected cells, is greater than the addition of the effect of administering the one or more artemisinin-related compounds by themselves and the effect of administering the one or more HDAC inhibitors by themselves. A synergistic effect can be calculated, for example, using suitable models/methods such as the highest single agent model [17], the Loewe additivity model [18], the Bliss independence model [19], the, the Chou-Talalay method [20], the Sigmoid-Emax equation [21], or the median-effect equation [22]. Various tools/software can be used to assess synergy, including, but not limited to, CompuSyn [23], Synergyfinder [24], Mixlow [25], COMBIA [26], MacSynergyII [27], Combenefit [28], Combinatorial Drug Assembler [29] (http://cda.i-pharm.org/), Synergy Maps [30] (http://richlewis42.github.io/synergy-maps/), DT-Web [31] (http://alpha.dmi.unict.it/dtweb/), and TIMMA-R [32].

In certain embodiments of the invention, the one or more artemisinin-related compounds and the one or more HDAC inhibitors may be used in combination with other anti-viral or anti-cancer therapeutic approaches (e.g., administration of an anti-viral or anti-cancer agent, radiation therapy, phototherapy or immunotherapy) directed to treatment of HPV-induced condition, treatment of HPV-induced lesions, and/or treating HPV-infected cells. For example, such methods can be used in prophylactic cancer prevention, prevention of cancer recurrence and metastases after surgery, and as an adjuvant of other traditional cancer therapy. Similarly, the subject methods of the invention may be combined with other antiviral therapies.

Thus, the subject methods of the invention may further include as optional ingredients one or more agents already known for their use in the inhibition of cancer or precancer cells, for added clinical efficacy. These agents include, but are not limited to, interleukin-2,5′-fluorouracil, nedaplatin, methotrexate, vinblastine, doxorubicin, carboplatin, paclitaxel (Taxol), cisplatin, 13-cis retinoic acid, pyrazoloacridine, and vinorelbine. Appropriate amounts in each case will vary with the particular agent, and will be either readily known to those skilled in the art or readily determinable by routine experimentation, methotrexate, vinblastine, doxorubicin, and cisplatin.

In certain cases, the methods of the invention may further include as optional ingredients one or more agents already known for their anti-viral effects, for added clinical efficacy. These agents include, but are not limited to, 5′-fluorouracil, interferon alpha, imiquimod, lamivudine, arsenic trioxide, capsaicin, nucleoside analogues (e.g., acyclovir), and antiviral vaccines.

### Kits Comprising Pharmaceutical Compositions and a Package Insert

An aspect of the invention relates to kits containing one or more pharmaceutical compositions of the present invention and a package insert or a means for delivery of the first and/or second pharmaceutical composition. As used herein, a “kit” is a commercial unit of sale, which may comprise a fixed number of doses of the one or more pharmaceutical compositions. By way of example only, a kit may provide a 30-day supply of dosage units of one or more fixed strengths, the kit comprising 30 dosage units, 60 dosage units, 90 dosage units, 120 dosage units, or other appropriate number according to a physician's instruction. Optionally, a kit may provide a 90-day supply of dosage units.

Optionally, provided herein is a kit comprising a first pharmaceutical composition, a second pharmaceutical composition, and a package insert, wherein the first pharmaceutical composition comprises artesunate; the second pharmaceutical composition comprises panobinostat or vorinostat; and a means for topical delivery of the first and second pharmaceutical composition to a cervix of a subject with a human papillomavirus (HPV)-induced condition.

In some embodiments, the kit may comprise a pharmaceutical composition comprising one or more artemisinin-related compounds according to the present invention, and a pharmaceutical composition comprising one or more HDAC inhibitors according to the present invention. In some embodiments, the kit may comprise a pharmaceutical composition comprising one or more artemisinin-related compounds and one or more HDAC inhibitors according to the present invention.

As used herein, “package insert” means a document which provides information on the use of the one or more pharmaceutical compositions, safety information, and other information required by a regulatory agency. A package insert can be a physical printed document in some embodiments. Alternatively, a package insert can be made available electronically to the user, such as via the Daily Med service of the National Library of Medicines of the National Institute of Health, which provides up-to-date prescribing information. (See https://dailymed.nlm.nih.gov/dailymed/index.cfm.)

In some embodiments, the package insert may inform a user of the kit that the one or more pharmaceutical compositions may be administered according to the methods and treating regimens of the present invention. In some embodiments, the package insert informs a user of the kit that the one or more pharmaceutical compositions can be co-administered with an anti-cancer or anti-viral therapy.

In certain embodiments, the kit comprises a means for delivery of the first and/or second pharmaceutical composition. For example, the kit can include a depot system, a patch, an intravaginal ring ring, syringe or applicator (e.g., CerviPrep) for topical delivery of a composition containing one or both pharmaceutical compositions to a target surface area of a subject with a human papillomavirus (HPV)-induced condition. For example, delivery means can be designed for a metered dosage of the one or more pharmaceutical compositions to the cervix of the subject.

### EXAMPLES

The invention now being generally described, it will be more readily understood by reference to the following examples, which are included merely for purposes of illustration of certain aspects and embodiments of the present invention, and are not intended to limit the invention.

### Example 1. Effect of an Artemisinin-Related Compound and HDAC Inhibitor Against Cervical Cancer

A study was conducted that examined the efficacy of an artemisinin-related compound and an HDAC inhibitor, alone and in combination, against cervical cancer model cell lines. For the study, the artemisinin-related compound was artesunate, and the HDAC inhibitor was panobinostat.

### Methods

To test the individual effects of artesunate and panobinostat, three cervical cancer cell model lines (Caski, SiHa, and HeLa cells) were obtained from the American Type Culture Collection (Manassas, Va.) and maintained in DMEM (Invitrogen) supplemented with 10% fetal bovine serum (Quality Biological, Inc., Gaitherburg, Md.) and penicillin/streptomycin (Invitrogen), in accordance with previous experiments [7]. The cells were split every 3 to 5 days to prevent any of the lines from becoming fully confluent.

To prepare the cells for treatment, 6000 cells per well were seeded in 96-well plates (for each cell line). Cells were then incubated for a 24-hour period to allow them to adhere to the wells. The cells were then treated with various concentrations of artesunate or panobinostat. For artesunate, the concentrations used for treating each cell type were 80 μM, 40 μM, 20 μM, 10 μM, 5 μM, 2.5 μM, and 1.25 μM. For panobinostat, the concentrations used for treating the Caski and HeLa cells were 256 nM, 128 nM, 64 nM, 32 nM, 16 nM, 8 nM, and 4 nM; and the concentrations used for treating SiHa cells were 512 nM, 256 nM, 128 nM, 64 nM, 32 nM, 16 nM, and 8 nM due to their increased resistance to the drug. Control wells consisted of DMEM, in which the DMSO concentration was the highest drug concentration (0.2% for artesunate, 0.064% for panobinostat) tested. Each concentration was performed in triplicate and the control was conducted in sextuplicate.

After an incubation time of 72 hours, the CELLTITER-GLO® Luminescent Cell Viability Assay (Promega) was conducted. The buffer/enzyme solution was mixed and applied to the cells according to the Promega protocol. Cells were then placed on the orbital shaker for 3 minutes, incubated for 30 minutes, and placed in the luminometer for reading.

To analyze the data, the background (from only the DMEM media) was subtracted from the luminescence of each well. The program GraphPad Prism 7 was then used to calculate the IC₅₀ values and the cell viability curves for each drug and cell line [33]. All readings were normalized based upon the control values.

To test the combined effects of artesunate and panobinostat, HeLa cells were cultured as previously described. The cells were then seeded in 96-well plates as discussed above. After the 24-hour incubation period, the cells were treated by the following means. Using the approximate IC₅₀ values of artesunate and panobinostat for HeLa cells, 5 μM and 10 nM, respectively, the following concentrations were tested: 1.25 μM, 2.5 μM, 5 μM, 10 μM, and 20 μM for artesunate; and 2.5 nM, 5 nM, 10 nM, 20 nM, and 40 nM for panobinostat. All possible pairings of each drug concentration were tested. All wells and the control had a DMSO concentration of 0.064%. All pairings were performed in triplicate and the control was performed in sextuplicate.

After 72 hours of incubation time, the CELLTITER-GLO® Luminescent Cell Viability Assay (Promega) was conducted as above. The combination effect of the drugs was analyzed using the CompuSyn program [23]. The background (DMEM only) was subtracted as described previously and all experimental wells were normalized to the average of the controls (as above).

### Results

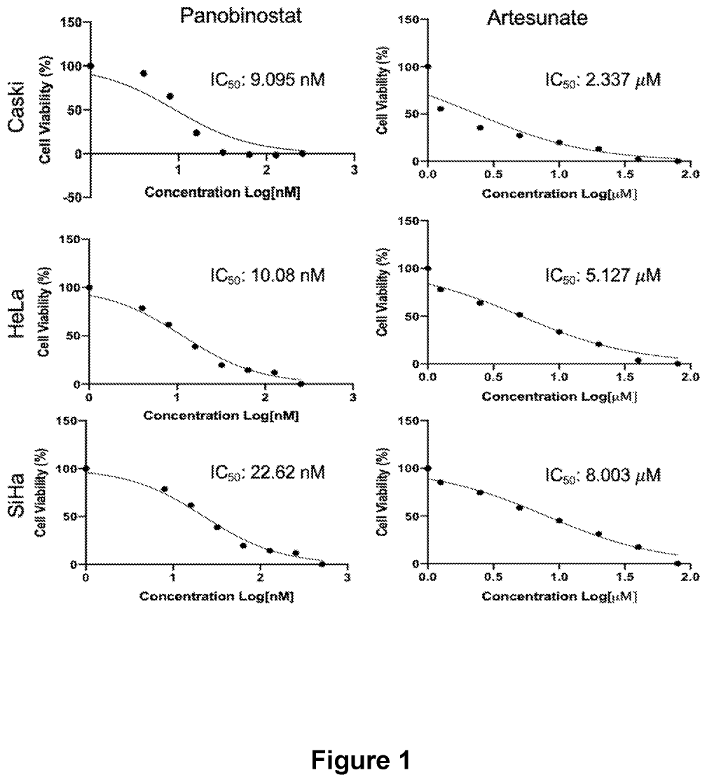

When tested individually, both artesunate and panobinostat were effective in inducing apoptosis in the cervical model cell lines. As shown in FIG. 1, the IC₅₀ values of panobinostat in all three cell lines were lower than the corresponding IC₅₀ values for artesunate. This is because all panobinostat values were on the order of nanomolar, while the artesunate values were on the order of micromolar. Additionally, as shown in FIG. 1, the IC₅₀ values of both panobinostat and artesunate were lowest in Caski cells, intermediate in HeLa cells, and highest in SiHa cells.

Further, as shown by Tables 1 and 2, the 95% confidence intervals for these IC₅₀ values did not overlap, indicating statistical significance. One exception was the confidence interval for the IC₅₀ values of panobinostat in Caski and HeLa cells (Table 1).

TABLE 1
IC₅₀ Confidence intervals for
artesunate in three cervical cancer model cell lines.

| Cell Line | IC₅₀ Confidence Interval (μM) | R² |
|---|---|---|
| Caski | (1.737, 3.135) | 0.8393 |
| HeLa | (4.332, 6.069) | 0.9552 |
| SiHa | (6.797, 9.427) | 0.9579 |

TABLE 2
IC₅₀ Confidence intervals for
panobinostat in three cervical cancer model cell lines

| Cell Line | IC₅₀ Confidence Interval (nM) | R² |
|---|---|---|
| Caski | (6.547, 12.52) | 0.8807 |
| HeLa | (7.710, 13.21) | 0.9349 |
| SiHa | (19.87, 25.71) | 0.9773 |

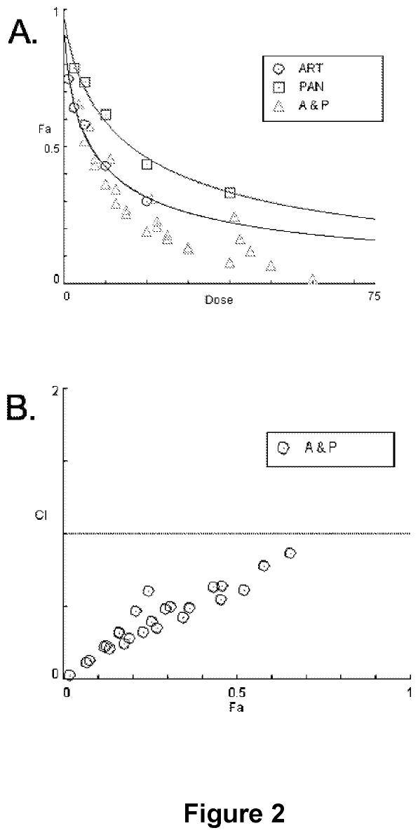

Artesunate and panobinostat each had lower IC₅₀ values in HeLa cells when combined rather than either drug alone (see FIG. 2A). For instance, alone, the IC₅₀ of artesunate was 5.127 μM and the IC₅₀ of panobinostat was 10.08 nM in HeLa cells (see FIG. 1). When combined, 2.5 μM of artesunate and 2.5 nM of panobinostat killed 47.7774% of HeLa cells (see Table 3). Further, as shown by FIG. 2B, all combinations of panobinostat and artesunate fell below the y=1 line of the combination index (CI) plot, which indicates that artesunate and panobinostat surprisingly functioned synergistically to kill HeLa cells.

TABLE 3
Effect of artesunate and panobinostat,
singly and in combination, on HeLa cells.

| Dose of Artesunate (uM) | Dose of Panobinostat (nM) | Effect |
|---|---|---|
| 1.25 | | 0.74571 |
| 2.5 | | 0.64184 |
| 5.0 | | 0.58082 |
| 10.0 | | 0.42868 |
| 20.0 | | 0.30154 |
| | 2.5 | 0.78639 |
| | 5.0 | 0.73792 |
| | 10.0 | 0.61772 |
| | 20.0 | 0.43589 |
| | 40.0 | 0.33215 |
| 1.25 | 2.5 | 0.65651 |
| 2.5 | 2.5 | 0.52226 |
| 5.0 | 2.5 | 0.43351 |
| 10.0 | 2.5 | 0.29600 |
| 20.0 | 2.5 | 0.20991 |
| 1.25 | 5.0 | 0.57931 |
| 2.5 | 5.0 | 0.45669 |
| 5.0 | 5.0 | 0.36443 |
| 10.0 | 5.0 | 0.25602 |
| 20.0 | 5.0 | 0.16275 |
| 1.25 | 10.0 | 0.45801 |
| 2.5 | 10.0 | 0.34721 |
| 5.0 | 10.0 | 0.27172 |
| 10.0 | 10.0 | 0.19224 |
| 20.0 | 10.0 | 0.12660 |
| 1.25 | 20.0 | 0.31166 |
| 2.5 | 20.0 | 0.23042 |
| 5.0 | 20.0 | 0.17750 |
| 10.0 | 20.0 | 0.13422 |
| 20.0 | 20.0 | 0.07661 |
| 1.25 | 40.0 | 0.24631 |
| 2.5 | 40.0 | 0.16249 |
| 5.0 | 40.0 | 0.12005 |
| 10.0 | 40.0 | 0.06701 |
| 20.0 | 40.0 | 0.01870 |

### Example 4. Effect of Artemisinin-Related Compounds and HDAC Inhibitors

To assess the effects of artemisinin-related compounds and HDAC inhibitors on cancer and precancerous conditions, Hela and Siha cells were selected representing cancerous conditions and HEC/16E6E7 cells were selected representing precancerous conditions. The cell lines were treated with artesunate, vorinostat, and panobinostat individually and in combination. The IC50 values were calculated from the cell viability assays for each drug alone and in combination.

To prepare the cells for treatment, 4,000 Hela or Siha cells or 8000 HEC cells per well were seeded in 96-well plates (for each cell line). Cells were then incubated for a 24-hour period to allow them to adhere to the wells. The cells were then treated with various concentrations of artesunate, panobinostat, and vorinostat.

For the experiments involving artesunate and panobinostat, the concentrations used varied by cell type. For artesunate, the concentrations used for treating Hela and Siha cell types were 40 μM, 20 μM, 10 μM, 5 μM, 2.5 μM, and 1.25 μM. For panobinostat, the concentrations used for treating Hela and Siha cell types were 80 μM, 40 μM, 20 μM, 10 μM, 5 μM and 2.5 μM. For the artesunate (ART) and panobinostat (PAN) combination in the Hela and Siha cell types, the concentrations were 40 and 80 μM, 20 and 40 μM, 10 and 20 μM, 5 and 10 μM, 2.5 and 5 μM, and 1.25 and 2.5 μM, respectively. For treating the HEC/16E6E7 cell type, the concentrations used for artesunate were 80 μM, 40 μM, 20 μM, 10 μ5 μM, 2.5 μM, and 1.25 μM and the concentrations used for panobinostat were 160 μM, 80 μM, 40 μM, 20 μM, 10 μM, 5 μM and 2.5 μM. For the artesunate (ART) and panobinostat (PAN) combination in the HEC/16E6E7 cell type, the concentrations used were 80 and 160 μM, 40 and 80 μM, 20 and 40 μM, and 20 μM, 5 and 10 μM, 2.5 and 5 μM, and 1.25 and 2.5 μM respectively.

After an incubation time of 72 hours, the CELLTITER-GLO® Luminescent Cell Viability Assay (Promega) was conducted. The buffer/enzyme solution was mixed and applied to the cells according to the Promega protocol. Cells were then placed on the orbital shaker for 2 minutes and then placed in the luminometer for reading.

To analyze the data, the background (from only the DMEM media) was subtracted from the luminescence of each well. The program GraphPad Prism 9 was then used to calculate the IC₅₀ values and the cell viability curves for each drug and cell line. All readings were normalized based upon the control values.

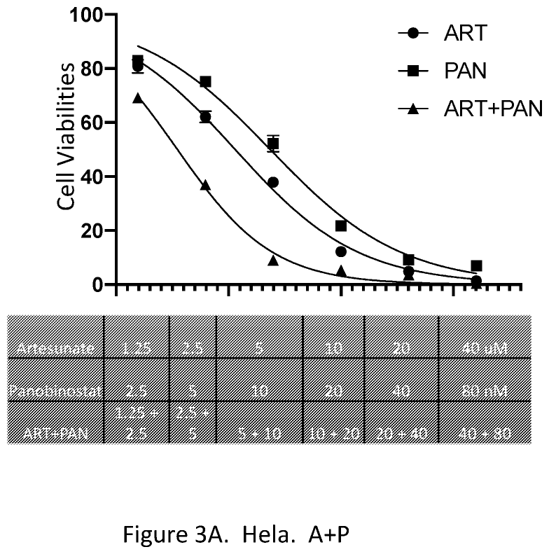

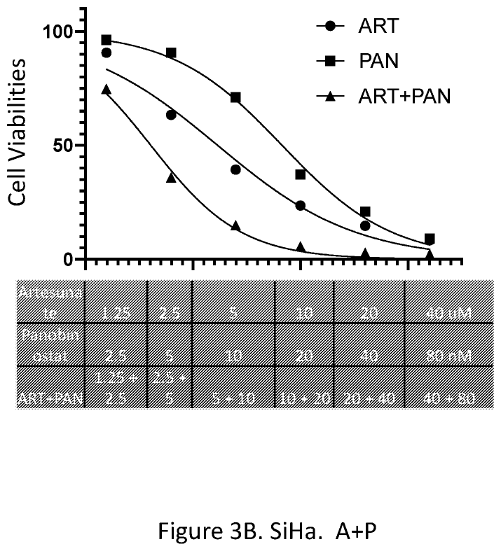

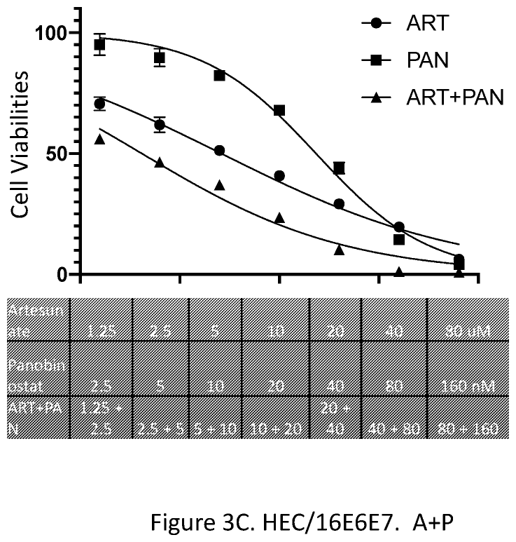

FIGS. 3A, 3B and 3C show dose-effect logarithmic curves of the percentage of cell viability after the respective treatment of each drug (panobinostat or artesunate) and the combination in each of the three cell lines (HeLa, SiHa and HEC/16E6E7). Analyses were conducted using GraphPad Prism 9. The combination index and dose-reduction index values were calculated based using the summary of the data for all three cell types. CI values of less than 1 indicate synergism. Dose reduction values are the measure of how many folds the dose of each drug in a synergistic combination may be reduced compared with the doses of each drug alone. By reducing dose, toxicity is reduced without compromising effect. The values are shown in Table 4 for IC₅₀ and in Table 5 for IC90.

TABLE 4
IC50 CI and DRI values for artesunate,
panobinostat and the combination.

| | | | ART + | ART + | | | |
|---|---|---|---|---|---|---|---|
| | | | PAN | PAN | | | |
| | ART | PAN | ART | PAN | DRI | DRI | |
| IC50 | (μM) | (μM) | (μM) | (μM) | ART | PAN | CI |
| Hela | 3.7 | 14.7 | 1.96 | 3.91 | 1.89 | 3.76 | 0.79 |
| Siha | 4.83 | 17.81 | 1.86 | 3.71 | 2.60 | 4.80 | 0.59 |
| HEC/16E6E7 | 4.93 | 25.04 | 2.4 | 4.81 | 2.05 | 5.21 | 0.68 |

TABLE 5
IC90 CI and DRI values for artesunate,
panobinostat and the combination.

| | | | ART + | ART + | | | |
|---|---|---|---|---|---|---|---|
| | | | PAN | PAN | | | |
| | ART | PAN | ART | PAN | DRI | DRI | |
| IC50 | (μM) | (μM) | (μM) | (μM) | ART | PAN | CI |
| Hela | 14.29 | 60.45 | 6.08 | 12.17 | 2.35 | 4.97 | 0.62 |
| Siha | 29.96 | 67.58 | 8.85 | 17.69 | 3.05 | 3.82 | 0.59 |
| HEC/16E6E7 | 77.57 | 114.92 | 13.08 | 26.16 | 5.93 | 4.39 | 0.4 |

For the experiments involving artesunate and vorinostat the concentrations used also varied by cell type. The methods of analysis are the same as those described above. For artesunate, the concentrations used for treating the Hela cell type were 40 μM, 20 μM, 10 μM, 5 μM, and 2.5 μM. For vorinostat, the concentrations used for treating the Hela cell type were 8 μM, 4 μM, 2 μM, 1 μM and 0.5 μM. For the artesunate (ART) and vorinostat (SAHA) combinations in the Hela cell type, the concentrations were 40 and 8 μM, 20 and 4 μM, 10 and 2 μM, 5 and 1 μM, and 2.5 and 0.5 μM, respectively. For treating the Siha and HEC/16E6E7 cells, the concentrations of artesunate used were 80 μM, 40 μM, 20 μM, 10 μM, 5 μM, and 2.5 μM and the concentrations of vorinostat used were 16 μM, 8 μM, 4 μM, 2 μM, 1 μM and 0.5 μM. For the artesunate and vorinostat combination in the Siha and HEC/16E6E7 cells, the concentrations were 80 and 16 μM, 40 and 8 μM, 20 and 4 μM, 10 and 2 μM, 5 and 1 μM, and 2.5 and 0.5 μM, respectively. A DMSO experimental stock was made in DMEM as a negative control.

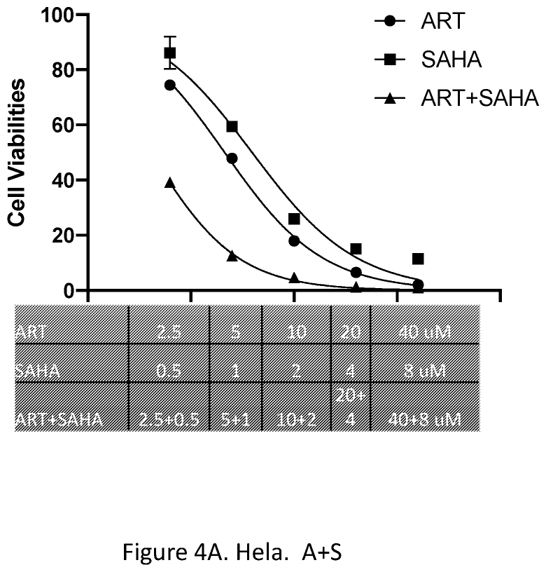

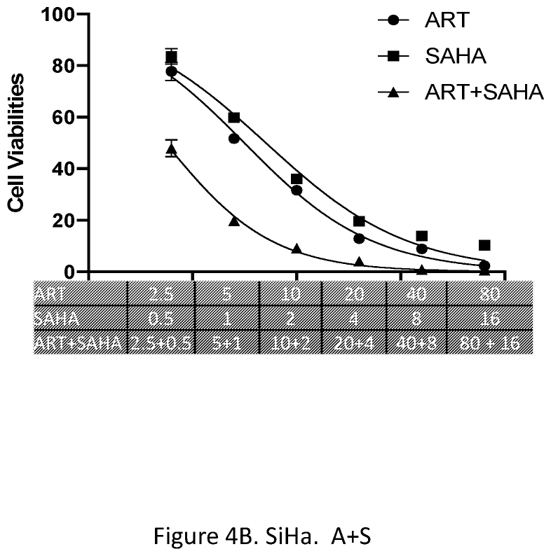

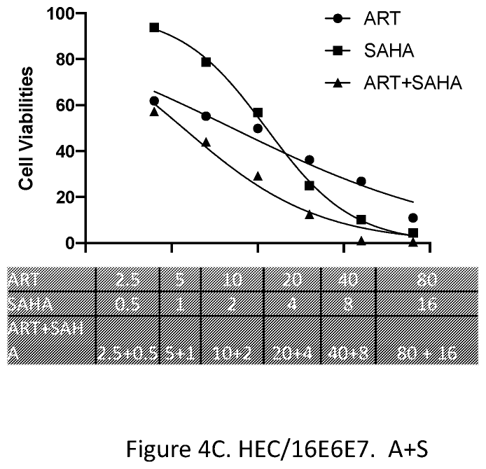

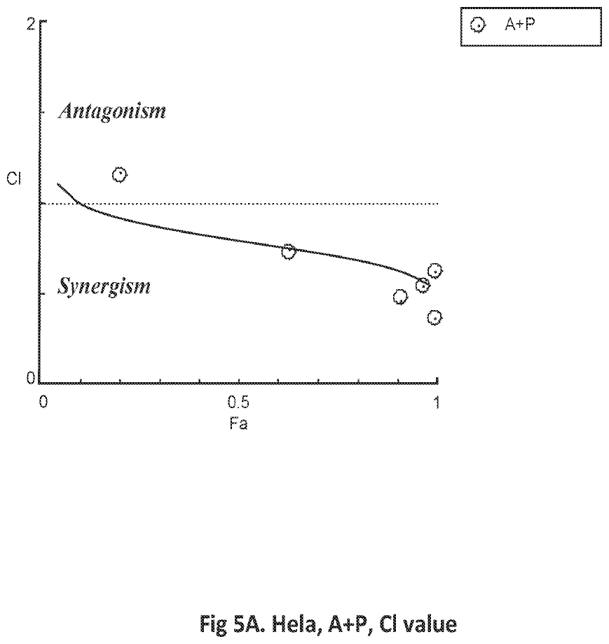

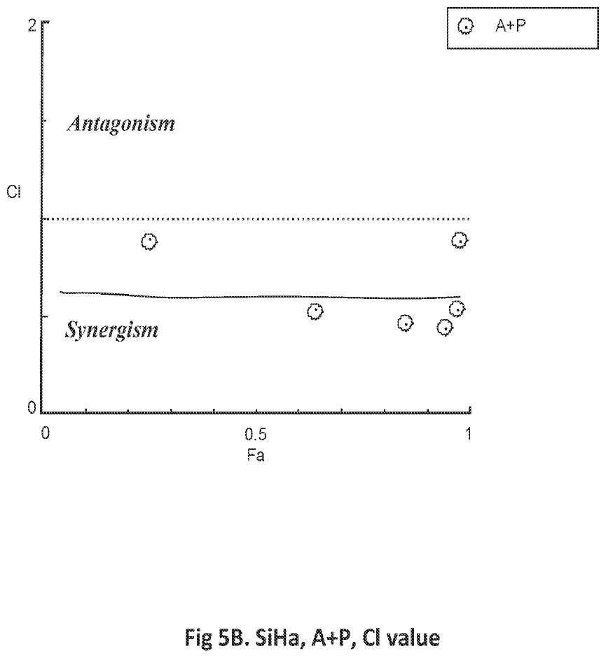

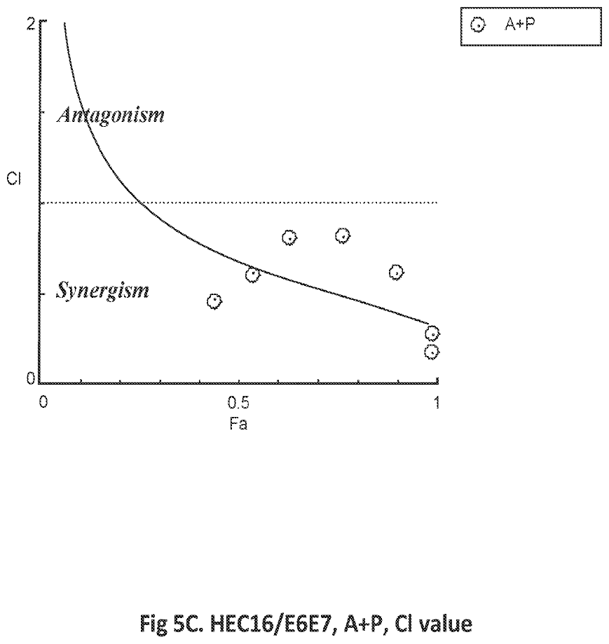

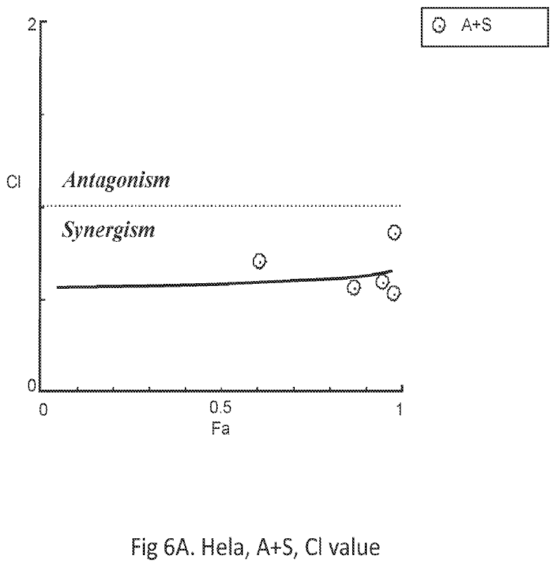

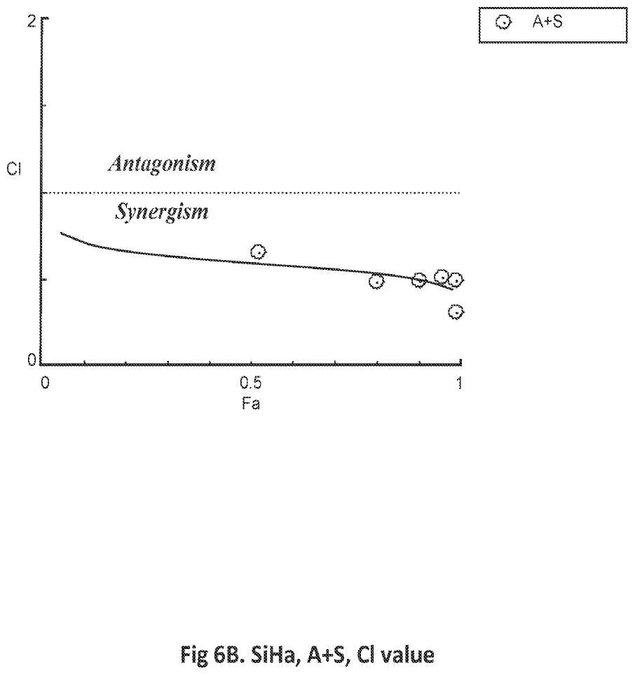

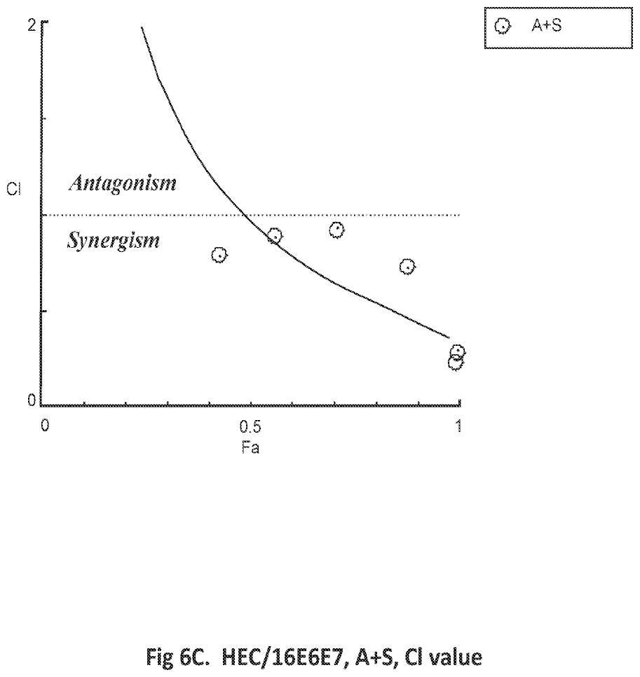

FIGS. 4A, 4B and 4C show dose-effect logarithmic curves of the percentage of cell viability after the respective treatment of each drug (vorinostat or artesunate) and the combination in each of the three cell lines (HeLa, SiHa and HEC/16E6E7). Analyses were conducted using GraphPad Prism 9. The combination index and dose-reduction index values were calculated based using the summary of the data for all three cell types. CI values of less than 1 indicate synergism. Dose reduction values are the measure of how many folds the dose of each drug in a synergistic combination may be reduced compared with the doses of each drug alone. By reducing dose, toxicity is reduced without compromising effect. The values are shown in Table 6 for IC₅₀ and in Table 7 for IC90.

TABLE 6
IC50 CI and DRI values for artesunate,
vorinostat (SAHA) and the combination.

| | | | ART + | ART + | | | |
|---|---|---|---|---|---|---|---|
| | | | SAHA | SAHA | | | |
| | ART | SAHA | ART | SAHA | DRI | DRI | |
| IC50 | (μM) | (μM) | (μM) | (μM) | ART | SAHA | CI |
| Hela | 4.55 | 1.38 | 1.6 | 0.32 | 2.84 | 4.31 | 0.58 |
| Siha | 6.06 | 1.81 | 2.44 | 0.48 | 2.48 | 3.77 | 0.67 |
| HEC/ | 6.99 | 2.31 | 4.21 | 0.84 | 1.66 | 2.75 | 0.97 |
| 16E6E7 | | | | | | | |

TABLE 7
IC90 CI and DRI values for artesunate,
vorinostat and the combination.

| | | | ART + | ART + | | | |
|---|---|---|---|---|---|---|---|
| | | | SAHA | SAHA | | | |
| | ART | SAHA | ART | SAHA | DRI | DRI | |
| IC50 | (μM) | (μM) | (μM) | (μM) | ART | SAHA | CI |
| Hela | 15.52 | 6.5 | 6.46 | 1.29 | 2.40 | 5.04 | 0.62 |
| Siha | 34.49 | 13.21 | 11.71 | 2.34 | 2.95 | 5.65 | 0.52 |
| HEC/ | 157.8 | 8.54 | 14.72 | 2.94 | 10.72 | 2.90 | 0.44 |
| 16E6E7 | | | | | | | |

The foregoing description is given for clearness of understanding only, and no unnecessary limitations should be understood therefrom, as modifications within the scope of the invention may be apparent to those having ordinary skill in the art.

Throughout this specification and the claims that follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

### REFERENCES

[1] World Health Organization. Human papillomavirus (HPV) and cervical cancer. (2018). http://www.who.int/news-room/fact-sheets/detail/human-papillomavirus-(hpv)-and-cervical-cancer.
[2] K. Münger et al., “Mechanisms of Human Papillomavirus-Induced Oncogenesis,” Journal of Virology, 2004, 78(21): 11451 LP-11460.
[3] Z. Zheng et al., “Papillomavirus genome structure, expression, and post-transcriptional regulation,” Frontiers in Bioscience, 2006, 11: 2286-2302.
[4] National Cancer Institute. Human Papillomavirus (HPV) Vaccines. (2018). https://www.cancer.gov/about-cancer/causes-prevention/risk/infectious-agents/hpv-vaccine-fact-sheet.
[5] Centers for Disease Control and Prevention. Human Papillomavirus (HPV). (2018). https://www.cdc.gov/hpv/parents/questions-answers.html.
[6] S. J. Hwang et al., “Biomarkers of cervical dysplasia and carcinoma,” Journal of Oncology, 2011, 2012: 507286.
[7] G. L. Disbrow et al., “Dihydroartemisinin Is Cytotoxic to Papillomavirus-Expressing Epithelial Cells In vitro and In vivo,” Cell and Tumor Biology, 2005, 65(23): 10854-10861.
[8] C. L. Trimble et al., “A First-In-Human Proof-of-Concept Trial of Intravaginal Artesunate to Treat Cervical Intraepithelial Neoplasia 2/3 (CIN2/3),” Gynecologic Oncology, 2020, 157(1): 188-194.
[9] A. C. West et al., “An Intact Immune System Is Required for the Anticancer Activities of Histone Deacetylase Inhibitors,” Cancer Research, 2013, 73(24): 7265-7276.
[10] M. Conte et al., “HDAC Inhibitors As Epigenetic Regulators for Cancer Immunotherapy,” The International Journal of Biochemistry & Cell Biology, 2018, 98: 65-74.
[11] D. D. Vo et al., “Enhanced Anti-Tumor Activity Induced By Adoptive T Cell Transfer and the Adjunctive Use of the HDAC Inhibitor LAQ824,” Cancer Research, 2009,
[12] A. F. Setiadi et al., “Epigenetic Enhancement of Antigen Processing and Presentation Promotes Immune Recognition of Tumors,” Cancer Research, 2008, 68(23): 9601-9607.
[13] Y.-L. Hong et al., “The Interaction of Artemisinin with Malarial Hemozoin,” Molecular and Biochemical Parasitology, 1974, 63(1): 121-128.
[14] D. L. Stout, “The Role of Transferrin in Heme Transport,” Biochemical and Biophysical Research Communications, 189(2): 765-770.
[15] A. Smith et al., “Expression of the Haemopexin-Transport System in Cultured Mouse Hepatoma Cells: Links Between Haemopexin and Iron Metabolism,” Biochemical Journal, 1988, 256(3): 941-950.
[16] A. Smith et al., “Hemopexin Joins Transferrin as Representative Members of a Distinct Class of Receptor-Mediated Endocytic Transport Systems,” European Journal of Cell Biology, 1990, 53(2): 234-245.
[17] M. C. Berenbaum, “What is Synergy?,” Pharmacological Reviews, 1989, 41(2): 93-141.
[18] S. Loewe, “The Problem of Synergism and Antagonism of Combined Drugs,” Arzneimittel-Forschung, 1953, 3(6): 285-290.
[19] C. I. Bliss, “The Toxicity of Poisons Applied Jointly,” Annals of Applied Biology, 1939, 26(3): 585-615.
[20] T.-C. Chou, “Drug Combination Studies and Their Synergy Quantification Using the Chou-Talalay Method,” Cancer Research, 2010, 70(2): 440-446.
[21] N. H. G. Holford et al., “Understanding the Dose-Effect Relationship: Clinical Application of Pharmacokinetic-Pharmacodynamic Models,” Clinical Pharmacokinetics, 1981, 6: 429-453.
[22] T. C. Chou et al., “Quantitative Analysis of Dose-Effect Relationships: The Combined Effects of Multiple Drugs or Enzyme Inhibitors,” Advances in Enzyme Regulation, 1984, 22: 27-55.
[23] T. Chou et al., CompuSyn for Drug Combinations: PC Software and User's Guide: A Computer Program for Quantitation of Synergism and Antagonism in Drug Combinations, and the Determination of IC₅₀ and ED50 and LD50 Values, 2005, ComboSyn, Paramus, N.J., USA.
[24] L. He et al., “Methods for Highthroughput Drug Combination Screening and Synergy Scoring,” BioRxiv, 2016.
[25] J. C. Boik et al., “An R package for Assessing Drug Synergism/Antagonism,” Journal of Statistical Software, 2010, 34(6): 1-18.
[26] M. Kashif, “Synergy/Antagonism Analyses of Drug Combinations,” R package version 104, 2015.
[27] M. Prichard et al., MacSynergy II. Version 1.0. User's Manual, 1993, University of Michigan, Ann Arbor, Mich., USA.
[28] G. Y. Di Veroli et al., “Combenefit: an Interactive Platform for the Analysis and Visualization of Drug Combinations,” Bioinformatics, 2016, 32(18): 2866-2868.
[29] J.-H. Lee et al., “CDA: combinatorial Drug Discovery Using Transcriptional Response Modules,” PLoS ONE, 2012, 7(8), Article ID e42573.
[30] R. Lewis et al., “Synergy Maps: Exploring Compound Combinations Using Network-Based Visualization,” Journal of Cheminformatics, 2015, 7(1), article 36, 11 pages.
[31] S. Alaimo et al., “DT-Web: a Web-Based Application for Drug-Target Interaction and Drug Combination Prediction Through Domaintuned Network-Based Inference,” BMC Systems Biology, 2015, 9(3): 1.
[32] L. He et al., “TIMMAR: an R package for Predicting Synergistic Multi-Targeted Drug Combinations in Cancer Cell Lines or Patient-Derived Samples,” Bioinformatics, 2015, 31(11): 1866-1868.
[33] Logarithmic Curve-Fit vs. Normalized Response Analysis Using GraphPad Prism Version 8.0.2 for macOS, GraphPad Software, San Diego Calif. USA, www.graphpad.com.

1. A method of treating a human papillomavirus (HPV)-induced condition in a human subject, the method comprising administering a therapeutically effective amount of one or more artemisinin-related compound and a therapeutically effective amount of one or more histone deacetylase (HDAC) inhibitors to the subject, wherein the administering treats the human papillomavirus (HPV)-induced condition in the human subject.

2. The method of claim 1, wherein the HPV-induced condition is a precancerous condition comprising precancerous cells or precancerous lesions.

3. The method of claim 1, wherein the HPV-induced condition is cervical dysplasia.

4. The method of claim 1, wherein the one or more artemisinin-related compounds is artesunate.

5. The method of claim 1, wherein the one or more HDAC inhibitors is panobinostat.

6. The method of claim 1, wherein the one or more HDAC inhibitors is vorinostat.

7. The method of claim 1, wherein the one or more artemisinin-related compound is artesunate and the one or more HDAC inhibitor is panobinostat.

8. The method of claim 1, wherein the one or more artemisinin-related compound is artesunate and the one or more HDAC inhibitor is vorinostat.

9. The method of claim 1, wherein the one or more artemisinin-related compounds and the one or more HDAC inhibitors are not administered systemically to the human subject.

10. The method of claim 9, wherein the one or more artemisinin-related compounds and the one or more HDAC inhibitors each are administered topically.

11. The method of claim 1, further comprising identifying the subject with an HPV-induced condition.

12. The method of claim 11, wherein the subject comprises precancerous cells or precancerous lesions.

13. The method of claim 1, wherein the effect of the one or more artemisinin-related compounds for treating the HPV-induced condition and the effect of the one or more HDAC-inhibitors for treating the HPV-induced condition are synergistic.

14. A kit comprising a first pharmaceutical composition, a second pharmaceutical composition, and a package insert, wherein:
the first pharmaceutical composition comprises artesunate;
the second pharmaceutical composition comprises panobinostat or vorinostat; and
a means for topical delivery of the first and second pharmaceutical composition to a cervix of a subject with a human papillomavirus (HPV)-induced condition.
